(19) United States
(12) Patent Application Publication
(10) Pub. No.: US 2017/0287206 A1
(43) Pub. Date: Oct. 5, 2017

(54) METHOD AND APPARATUS FOR PROCESSING THREE-DIMENSIONAL IMAGE DATA

(71) Applicant: BERGEN TEKNOLOGIOVERFØRING AS, Bergen (NO)

(72) Inventors: Veronika Solteszova, Bergen (NO); Ivan Viola, Wien (AT); Åsmund Birkeland, Bergen (NO); Stefan Bruckner, Bergen (NO)

(73) Assignee: BERGEN TEKNOLOGIOVERFØRING AS, Bergen (NO)

(21) Appl. No.: 15/508,101

(22) PCT Filed: Sep. 2, 2015
(86) PCT No.: PCT/GB2015/052534

§ 371 Date: Mar. 1, 2017

(30) Foreign Application Priority Data
Sep. 2, 2014 (GB) 1415534.5

(51) Int. Cl. G06T 15/08; A61B 8/00

(52) U.S. Cl. CPC G06T 15/08; A61B 8/466; G06T 2200/04; G06T 2200/08

(57) ABSTRACT

A method of processing three-dimensional image data for volume-rendering from a viewpoint is described. Lower- and upper-bound-generating functions are used (7) to determine whether, across all possible values for the image voxels between respective lower and upper bounds, for each voxel (i) the voxel may be at least partially opaque under the opacity transfer function (8); and (ii) the voxel may be unoccluded from the viewpoint (9). A predetermined processing operation is then applied to these potentially-visible voxels, for which both determinations hold true (10) and the processed voxels may be rendered (11). The bound-generating functions and the processing operation are such that, for any three-dimensional image data, the value of a voxel after the processing operation will necessarily lie between the lower and upper bounds for that voxel.

This invention relates to methods and apparatus for processing three-dimensional image data, including, but not limited to, medical ultrasonography data.

Three-dimensional (3D) image data can be obtained from a variety of sources, including medical ultrasound scanners, sonar and radar. Ultrasonography is now a standard tool in obstetrics, cardiology, gastroenterology, and many other medical fields. The technology has progressed rapidly from initial one-dimensional (1D) signals, through standard two-dimensional (2D) sonography, to 3D volumetric ultrasound. In echocardiography, for example, ultrasound can be used to diagnose heart contraction efficiency and functionality of the valves.

3D image data typically comprises a regularly-spaced array of sampled intensity values, known as voxels. The value of a voxel typically corresponds to some property of matter located at, or adjacent to, the corresponding point in the space being imaged. For example, the voxel may represent the degree of ultrasound-wave reflection of biological tissue at that point.

In order to present 3D image data in a meaningful form to a human, such as a physician, it is typically necessary to render the 3D image as a two-dimensional (2D) view. This will typically involve certain voxels being rendered partially or completely transparent, in order that a region or structure of interest located within the volume of the image data will be visible in the 2D view. The decision as to what to which voxels should be visible may be made by applying an opacity transfer function to the image data.

Rapid increases in processing power, coupled with advances in digital beamforming, have enabled the real-time capture of high-resolution 3D volumes, enabling real-time motion imaging and making ultrasound a true “four-dimensional (4D)” imaging modality. 4D ultrasound offers many potential benefits in the prenatal diagnosis of neurological problems and in assessing heart defects.

However, despite its advantages, the useful visualization of ultrasound data is not straightforward. Compared with other common tomographic imaging methods such as CT and MRI, ultrasound has a poor signal-to-noise ratio and suffers from various acoustic artefacts such as attenuation, focusing and interference. In volume visualization these are particularly problematic as artefacts can obscure relevant structures and hence affect the diagnostic value of the resulting image.

Image data may therefore be filtered before it is rendered; for example, to remove speckle noise, or to enhance contrast. Such filtering is, however, computationally intense. For static 3D data or pre-recorded 4D sequences, filtering can be applied as a pre-processing step, before viewing, and is therefore not performance critical. A complex filtering operation may take several seconds to process the whole volume, but need only be performed once (typically during loading of the data). However, when data are streamed live at many volumes per second, and are to be rendered in real-time, e.g., during a medical examination, it may not then be possible to apply high-quality filtering, resulting in inferior images.

The present invention seeks to provide a faster approach to processing (e.g. filtering) 3D image data.

From a first aspect, the invention provides a computer-implemented method of processing three-dimensional image data comprising a plurality of voxels to generate processed image data suitable for volume rendering, from a viewpoint, using an opacity transfer function, wherein the method comprises:
applying a lower-bound-generating function to the image data to generate a lower bound for each voxel in the image data;
applying an upper-bound-generating function to the image data to generate an upper bound for each voxel in the image data;
for each voxel in the image data, determining whether there exists:
   (i) a value, between the lower bound and the upper bound for that voxel, at which the voxel is not completely transparent under the opacity transfer function; and
   (ii) a set of values for a set of sample points lying along or adjacent a viewing ray between the viewpoint and said voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray; and
applying a predetermined processing operation to those voxels for which both determinations are true, to generate processed image data,
   wherein the bound-generating functions and the predetermined processing operation are such that, for any three-dimensional image data, the value of a voxel after the processing operation is applied to the image data will always be between the lower bound and the upper bound given by the bound-generating functions when applied to that voxel.

From a second aspect, the invention provides an apparatus comprising processing means or logic configured:
to receive three-dimensional image data comprising a plurality of voxels;
to apply a lower-bound-generating function to the image data to generate a lower bound for each voxel in the image data;
to apply an upper-bound-generating function to the image data to generate an upper bound for each voxel in the image data;
for a predetermined opacity transfer function and a predetermined viewpoint, to determine, for each voxel in the image data, whether there exists:
   (i) a value, between the lower bound and the upper bound for that voxel, at which the voxel is not completely transparent under the opacity transfer function; and
   (ii) a set of values for a set of sample points lying along or adjacent a viewing ray between the viewpoint and said voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray; and
to apply a predetermined processing operation to those voxels for which both determinations are true, to generate processed image data suitable for volume rendering from the viewpoint,
   wherein the bound-generating functions and the predetermined processing operation are such that, for any three-dimensional image data, the value of a voxel after the processing operation is applied to the image data will always be between the lower bound and the upper bound given by the bound-generating functions when applied to that voxel.

From a third aspect, the invention provides software, or a signal or tangible medium bearing the same, comprising instructions which, when executed by processing means or logic, causes the processing means or logic:
to apply a lower-bound-generating function to three-dimensional image data, comprising a plurality of voxels, to generate a lower bound for each voxel in the image data;
to apply an upper-bound-generating function to the image data to generate an upper bound for each voxel in the image data;
for a predetermined opacity transfer function and a predetermined viewpoint, to determine, for each voxel in the image data, whether there exists:
   (i) a value, between the lower bound and the upper bound for that voxel, at which the voxel is not completely transparent under the opacity transfer function; and
   (ii) a set of values for a set of sample points lying along or adjacent a viewing ray between the viewpoint and said voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray; and
to apply a predetermined processing operation to those voxels for which both determinations are true, to generate processed image data suitable for volume rendering from the viewpoint,
   wherein the bound-generating functions and the predetermined processing operation are such that, for any three-dimensional image data, the value of a voxel after the processing operation is applied to the image data will always be between the lower bound and the upper bound given by the bound-generating functions when applied to that voxel.

Thus it will be seen by those skilled in the art that, in accordance with the invention, the predetermined processing operation (e.g. a filtering operation, such as a noise-reducing filter) is applied to those voxels that have the possibility of being visible after the processing operation; that is, to all those voxels that will not definitely be completely transparent after the processing operation and will not definitely be completely occluded (i.e., obscured from view) by other voxels after the processing and rendering operations. This is achieved by use of the bound-generating functions. The set of voxel for which both determinations are true is referred to as the set of “potentially visible voxels”.

The operation of determining whether there exists a value, between the lower and upper bounds, at which a voxel is not completely transparent, can be seen as equivalent to the operation of determining whether the voxel is completely transparent for all possible values between the lower and upper bounds. The first determination is true whenever the second is false, and vice versa.

The operation of determining whether there exists a set of values for the set of sample points, between respective lower and upper bounds, for which a voxel is not completely occluded, can be seen as equivalent to the operation determining whether the voxel is completely occluded for all possible values of the sample points between the respective lower and upper bounds for each sample point. The first determination is true whenever the second is false, and vice versa.

The bound-generating functions are preferably such that evaluating both functions for a voxel is substantially quicker than applying the processing operation to the voxel; e.g., taking fewer than half, or a tenth, of the number of processor clock cycles. Thus, the bound-generating functions can be applied to the whole image data in much less time than if the relatively-slow processing operation were to be applied to the whole image data. Instead, the processing operation can be reserved for those voxels for which the processing may have an effect on the resulting processed image data.

This approach allows for improved efficiency by enabling the processing operation to be evaluated for only certain voxels, rather than being applied indiscriminately to the entire image data set. The processing operation is thus preferably not applied to at least one, and preferably not to a majority, of the voxels for which one or both of the determinations (i) and (ii) are false. In preferred embodiments, the processing operation is applied only to those voxels for which both determinations are true, or only to those voxels for which both determinations are true and to a band of voxels surrounding these voxels. The band may be of uniform radius around the voxels for which both determinations are true. This radius may be determined by the processing operation. The use of such a band may be relevant where the processing operation comprises two or more stages, and where the values of voxels in the band can affect the results of a first stage of the processing operation, which can then affect the results of a second stage when the operation is applied to a voxel for which both determinations are true.

The processing operation is preferably such that its output at any given position depends only on values in a finite neighbourhood around that position; i.e., the processing operation preferably has compact support. One, or preferably both, of the lower-bound-generating function and the upper-bound-generating function are preferably such that their output at any given position depends only on values in the same finite neighbourhood around that position. In a preferred set of embodiments, the lower-bound-generating function, applied to a position, outputs the lowest value in a finite neighbourhood around that position, and/or the upper-bound-generating function, applied to a position, outputs the highest value in the finite neighbourhood around that position. The processing operation preferably uses the same shaped and sized neighbourhood at every position. It will be appreciated that determining the maximum and minimum values in a neighbourhood is a very quick operation, and that embodiments that use these bound-generating functions can therefore provide particularly large performance improvements compared with trying to apply the processing operation to every voxel in the image data.

In a preferred set of embodiments, the neighbourhood is cuboid; more preferably cubic. The minimum value in the respective neighbourhood of each voxel may then be determined particularly efficiently by first calculating, for each voxel in the image data, the minimum value along an x axis passing through that voxel over an interval equal to the width of the neighbourhood in the x direction. Then, for each voxel, the results of the x-direction calculations can be processed to calculate respective minimum values from the x-direction results along a y axis passing through each voxel, over an interval equal to the width of the neighbourhood in the y direction. Finally, for each voxel, the results of the y-direction calculations can be processed to calculate respective minimum values from the y-direction results along a z axis passing through each voxel, over an interval equal to the width of the neighbourhood in the z direction. This results in a substantially efficiency saving compared with calculating the minimum for each neighbourhood independently. For a cubic neighbourhood of radius r, this three-pass algorithm requires, on average, only 3×(2r−1) voxels to be processed in order to determine the minimum value for a given voxel, instead of processing the entire neighbourhood of (2r)³ voxels for each voxel. The maximum value is preferably determined by the same process, except for finding the maximum values, instead of the minimum values, in each direction.

Other, more complicated bound-generating functions, may be required where the processing operation can output a wider range of values than between the minimum and maximum values within a neighbourhood, and may still provide some benefit, however; for example, a lower-bound-generating function might output half of the lowest value in a neighbourhood, or an upper-bound-generating function might output twice the highest value in a neighbourhood.

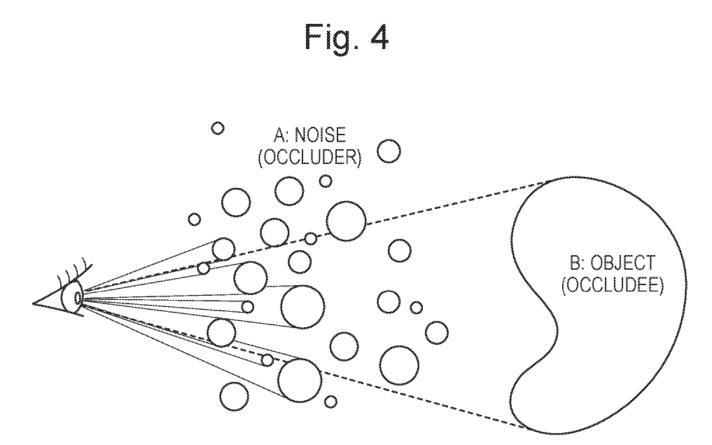

Importantly, this novel approach allows for the fact that processing operations such as filtering can themselves influence the visibility of other parts of the image data, because they modify the underlying data values. A naïve approach might be to determine which voxels are visible in a rendered image and then apply a processing operation to only those voxels. However, this is not guaranteed to produce a resulting image that is identical to the image that would have been obtained by processing the entire image data and then rendering the image. This can be seen by considering the situation shown in FIG. 4, in which noise blobs in the 3D image data are occluding an object of interest. If a noise-removing filtering operation were applied only to those voxels that are visible from the viewpoint, those parts of the object of interest that are occluded by blobs will not be filtered; however, the noise-removing filtering operation may remove at least some of the occluding blobs, thereby exposing the unfiltered object data in the rendered view. By contrast, the present approach can be shown always to give the same rendered image as would be obtained if the processing operation were applied to the entire image data.

The opacity transfer function outputs an opacity value for a given voxel value. The opacity value determines whether a voxel will be completely transparent, partially transparent, or completely opaque in the output of a volume rendering operation. For example, an output of 0 may signify complete transparency; and output of 1 may signify complete opacity; and a value in between may signify partial transparency/opacity. The opacity transfer function may be used to determine the visibility of a voxel along a viewing ray by accumulating the opacity values of sample points spaced along or adjacent to the viewing ray between the view point and the voxel, and determining whether this exceeds a visibility threshold. This visibility threshold may be the same value as the opacity transfer function outputs to signify complete opacity for a voxel; e.g. 1.

The particular opacity transfer function can be selected depending on what information in the image data should be rendered; e.g. what tissue types or boundaries are to be displayed when rendering an ultrasound scan of the human body for display on a two-dimensional screen.

The method may comprise explicitly calculating a value for a particular voxel, between the lower and upper bounds for the voxel, at which the voxel is not completely transparent under the opacity transfer function. However, in general, it is not necessary for this value to be determined explicitly, so long as it can be determined whether or not such a value exists. In a preferred set of embodiments, this determination is made for a particular voxel by determining the maximum opacity value attained by the opacity transfer function across the interval between the lower bound and the upper bound for the voxel, and determining whether the voxel is completely transparent with this opacity value. If the maximum is zero, then it can be concluded that this voxel would be completely transparent, even after the processing operation were applied to the image data. This operation can be made faster by pre-computing a set (e.g. a table) of maximum opacity values for a set of possible voxel value intervals. Maximum opacity values are preferably calculated for every different interval. For example, if the opacity transfer function is defined as having a particular value in each of n distinct ranges of voxel values (e.g. across 8 contiguous ranges), the method preferably comprises calculating the maximum opacity value of the opacity transfer function for each of the n×n intervals that starts with one of ranges 1 to n and ends in an equal or higher range 1 to n. These values may be stored in memory and used subsequently when processing each voxel in the image data.

Similarly, the method may comprise explicitly calculating a set of values, for a set of sample points along a viewing ray to a particular voxel, for which values the voxel is not completely occluded under the opacity transfer function. However, in general, it is not necessary for these values to be determined explicitly, so long as it can be determined whether or not such a set of values exists for each voxel. In a preferred set of embodiments, this determination is made for a particular voxel by accumulating opacity values of sample points along a viewing ray to the voxel, where each opacity value is the minimum opacity value attained by the opacity transfer function across the interval between the lower bound and the upper bound for the sample point, and determining whether the voxel is not completely occluded with these accumulated opacity values. This operation can be made faster by pre-computing a set (e.g. a table) of minimum opacity values for a set of possible sample-point value intervals. Minimum opacity values are preferably calculated for every different interval. For example, if the opacity transfer function is defined as having a particular value in each of n distinct ranges of voxel values (e.g. across 8 contiguous ranges), the method preferably comprises calculating the minimum opacity value of the opacity transfer function for each of the n×n intervals that starts with one of ranges 1 to n and ends in an equal or higher range 1 to n. These values may be stored in memory and used subsequently when processing each voxel in the image data.

Some or all of sample points lying along or adjacent a viewing ray may coincide with voxels from the image data. However, some may lie between voxels. In this case, the value at a sample point may be determined based on the values of one or adjacent or surrounding voxels; e.g. using trilinear interpolation. The sample points are preferably uniformly spaced along the viewing ray. This spacing may depend on the spacing between voxels in the image data along a Cartesian axis having the smallest angle to the viewing ray (i.e. the axis most nearly parallel to the viewing ray); in some embodiments it is approximately half the spacing between voxels, or it may be less than half the spacing.

More generally, the skilled person will appreciate that operations on the image data, such as applying a bound-generating function, or a processing operation, or an opacity transfer function, may be applied to, and make use of, the sampled voxels in the image data, but they may also, in some embodiments, be applied to, and/or make use of, interpolated values lying between sample positions; e.g., values generated using trilinear interpolation or other suitable techniques.

The image data may comprise regularly or irregularly spaced samples. The samples may represent electro-magnetic radiation intensity values, sound intensity values, and/or tissue density (e.g. from ultrasound or x-ray signals), or any other appropriate measurand. The applicant has recognised that the invention can be particularly well suited to processing 3D ultrasonography data. In one set of preferred embodiments, therefore, the image data comprises ultrasonography data. The apparatus may comprise an ultrasound scanner. Embodiments of the method may comprise receiving or collecting ultrasonography image data.

Although it is envisaged that, in preferred embodiments, every voxel in an image data set will normally be processed as described herein, it is possible that there may be embodiments or occasions in which certain voxels in an image data set are, for some reason, excluded from some or all of the processing steps described herein. For example, if a user “zooms in” to investigate small details in a rendered image, large parts of the volume may simply lie outside of the viewing frustum and therefore not need to be processed. In some embodiments, a cropping box may be used when determining the set of potentially visible voxels; e.g. to exclude parts of the image data that do not need to be rendered. Similarly, a clipping plane may be used to remove unwanted parts of the data, e.g. by applying a clipping membrane as an image containing depth values when determining the set of potentially visible voxels.

The processing operation may comprise any suitable filtering operation (i.e. an operation to remove one or more unwanted components or features from the image data). In some embodiments, the processing operation may comprise any one or more of the following: a smoothing filter, a noise-reducing filter, a mean filter, a median filter, a Gaussian smoothing filter, a contrast-enhancing filter, a Perona-Malik anisotropic diffusion, bilateral filtering, etc. It may comprise a segmentation operation; for example, it may comprise the real-time segmentation of vessels in ultrasonography data, possibly using principal component analysis in each neighbourhood of a point or voxel. The benefits may not be so great when certain simple filtering operations, such as mean filtering or Gaussian filtering, are applied on their own, but such operations may also be combined with other processing in some embodiments.

The processed image data may be stored in a memory. It may be volume rendered from the viewpoint using the opacity transfer function. The processed image data may be displayed, e.g. on a two-dimensional (2D) display screen (potentially after further processing of the data, such as adding shadows or colour), or may be transmitted over a data connection (e.g. to a server or storage medium), or may be processed further, or any combination of these. Apparatus embodying the invention may comprise a display screen for displaying a rendered view derived from the processed image data.

Because the method enables fast filtering and rendering of 3D image data, it is particularly well suited to processing moving 3D image data in real-time (e.g. so-called “4D” ultrasound scanning). The invention therefore extends to a method of processing moving three-dimensional image data, comprising applying steps according to any of the aspects or embodiments described herein to a sequence of two or more frames of three-dimensional image data. The processed frames are preferably displayed as a rendered video display. The display preferable occurs in real time or substantially real time. The method preferable comprises displaying a 2D rendered frame generated from one image-data set at the same time as processing a later image-data set in the sequence (e.g. the next frame in the sequence). Successive image data sets (frames) are preferably processed at a rate of at least one per second, and more preferably at a rate of around 15 or more frames per second.

Any suitable processing means or logic may be configured to implement some or all steps of embodiments of the invention. This may take various different forms. It may comprise one or more of: a central processing unit, a graphics processing unit, a microcontroller, an ASIC and an FPGA. Processing may be carried out on a single device or may be shared across multiple devices in any appropriate way. For instance, one device may determine whether each voxel in the image data is potentially visible, and a different device (possibly remote from the first) may apply the processing operation to those voxels. In some embodiments, sampled image data may be collected by a first device (e.g. an ultrasound scanner) and sent to one or more remote computers or servers for carrying out one or more of the steps described herein.

Features of any aspect or embodiment described herein may, wherever appropriate, be applied to any other aspect or embodiment described herein. Where reference is made to different embodiments or sets of embodiments, it should be understood that these are not necessarily distinct but may overlap.

Certain preferred embodiments of the invention will now be described, by way of example only, with reference to the accompanying drawings, in which:

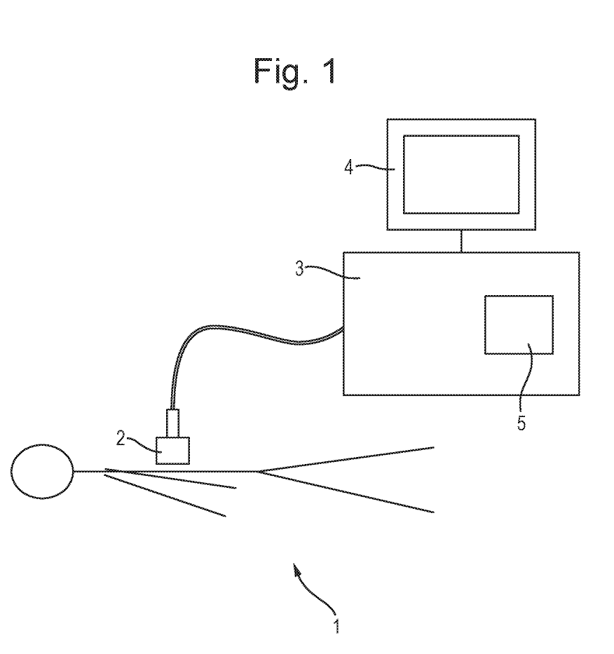

FIG. 1 is a figurative diagram showing apparatus embodying the invention being used on a patient;

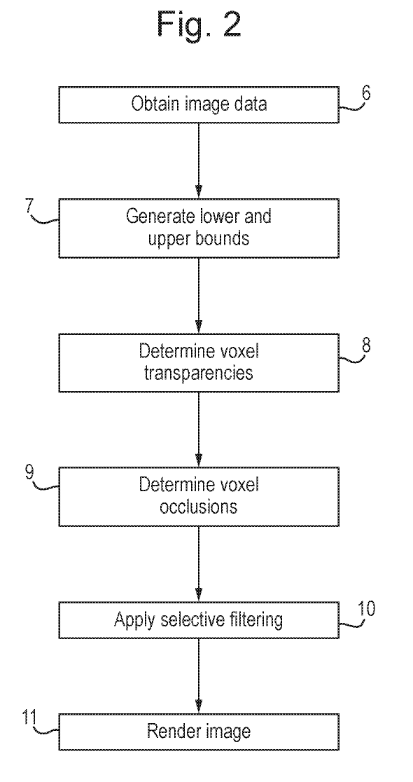

FIG. 2 is a flow chart illustrating major steps in an embodiment of the invention;

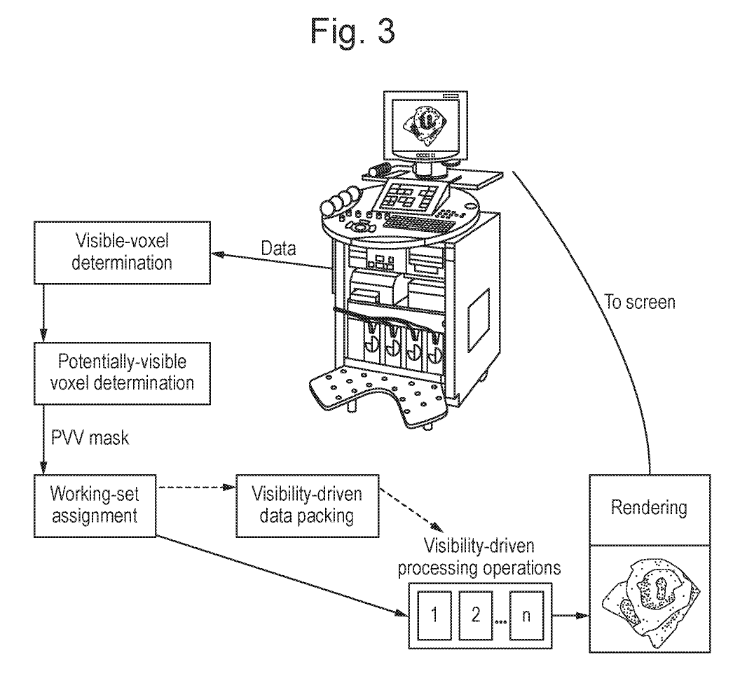

FIG. 3 is figurative diagram showing the outline processing pipeline for an embodiment of the invention;

FIG. 4 is a figurative diagram illustrating occlusion of an object from a viewpoint;

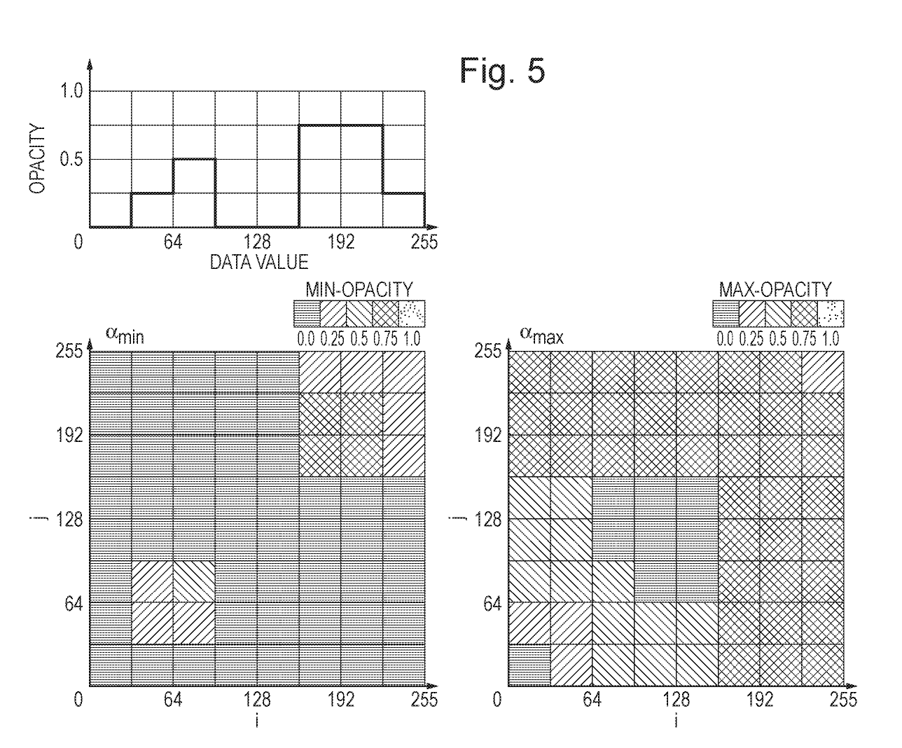

FIG. 5 is a plot of an exemplary opacity transfer function, and minimum and maximum tables derived therefrom;

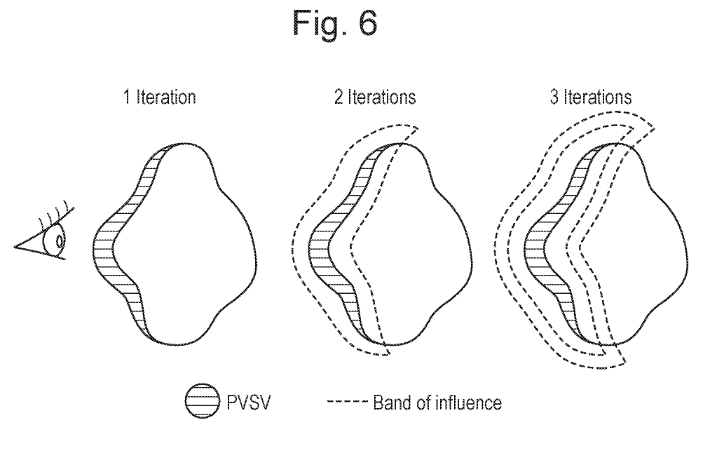

FIG. 6 is a figurative diagram illustrating a band of influence are a set of potentially visible voxels;

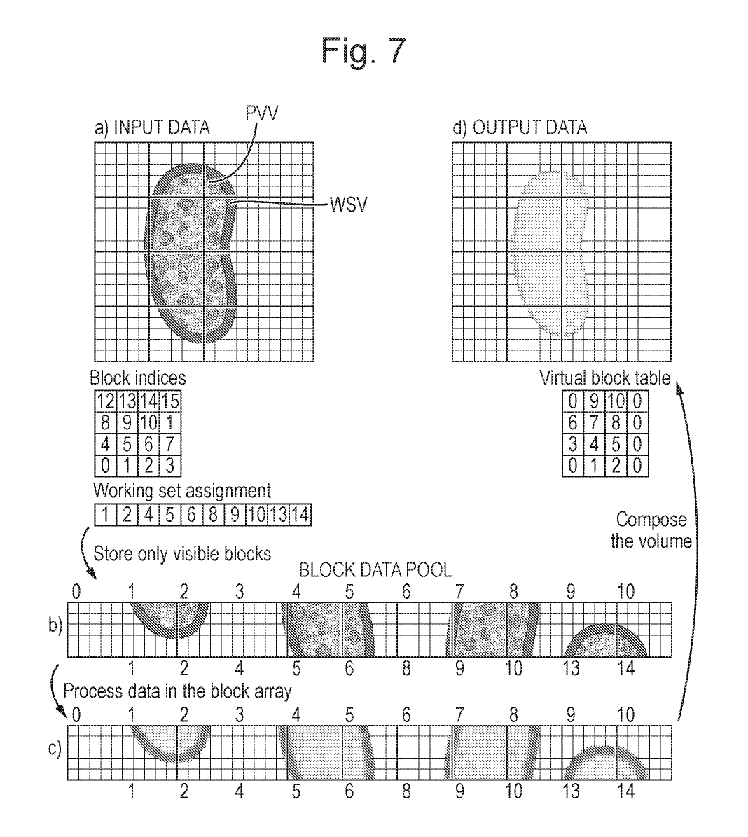

FIG. 7 is a diagram showing an example of two-dimensional data packing;

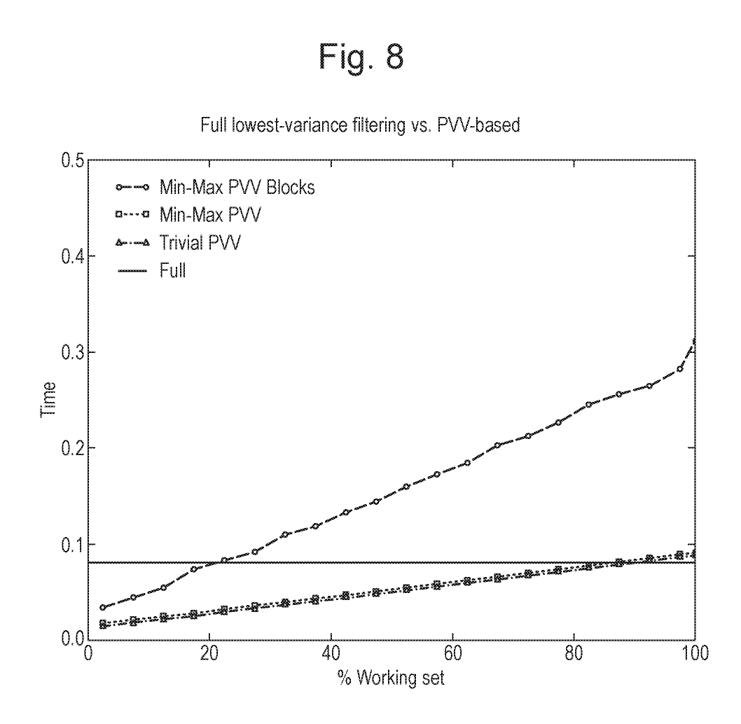

FIG. 8 is a chart showing performance of full lowest-variance filtering vs. PVV-based filtering; and

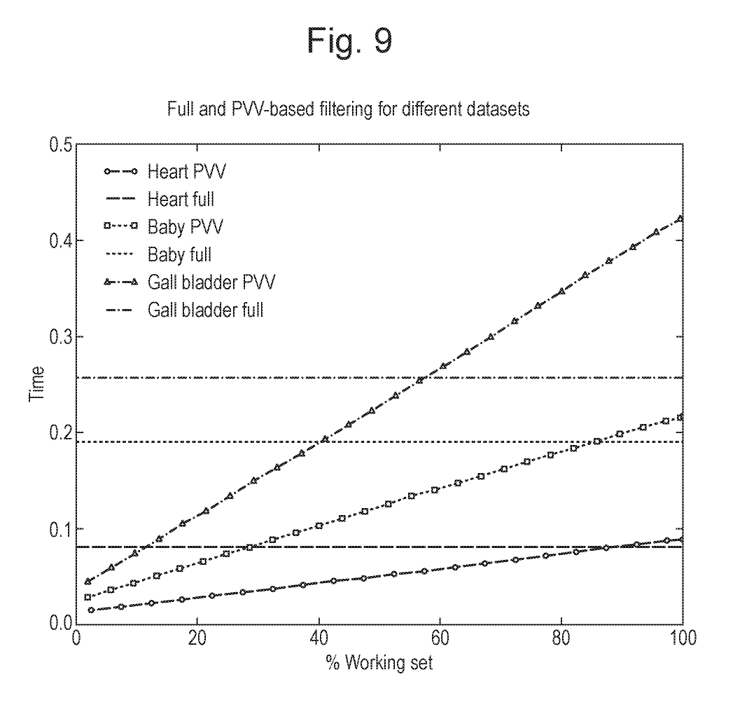

FIG. 9 is a chart showing performance of full and PVV-based filtering for different datasets.

FIG. 1 shows apparatus embodying the invention in use on a human patient 1. An ultrasound scanner handset 2 is directed towards the patient's heart. A processing unit 3 controls the transmission of ultrasound signals (e.g. pulses) from the ultrasound scanner handset 2, and also receives ultrasound reflection signals from the handset 2 for processing. The processing unit 3 applies standard processing steps known from conventional 4D ultrasonography. However, it also applies a filtering operation to each sequential 3D image frame as described herein. Because the filtering operation is applied only to selected voxels from each image frame, it can be applied in substantially real-time (ignoring buffering). The processing unit 3 applies a volumetric rendering operation to the filtered image data in order to display live video of selected regions or structures within the patient's heart on a two-dimensional display screen 4.

An operator may use input means 5, such as a button or a keyboard or a tracker ball to control the processing and rendering operations. For example, an operator may change the viewpoint for the rendering operation, or zoom in on a region of interest, or change the intensity of the filtering operation, or select different regions or structures to be rendered (e.g. by causing a change to the opacity transfer function).

FIG. 2 show some of the main steps carried out by the processing unit 3. It will be appreciated that some embodiments, some or all of these steps may be carried out by one or more remote servers (not shown), which may be communicably connected to the processing unit 3, e.g. by a computer network.

In outline, these steps include:
a first step 6 of obtaining or generating a 3D image data set to be rendered from signals received from the ultrasound scanner handset 3 (the data set may first be cropped or otherwise limited, depending on what parts of it are to be rendered);
a second step 7 of generating a lower bound and an upper bound for each voxel in the image data set, as described in more detail below;
a third step 8 of determining, for each voxel in the image data set, whether there is a value for the voxel, between the voxel's lower and upper bounds, for which the voxel is not completely transparent;
a fourth step 9 of determining, for each voxel in the image data set, whether there are values for the sample points along a viewing ray to the voxel, between their respective lower and upper bounds, for which the voxel is not completely occluded;
a fifth step 10 of applying a filtering (or other) operation selectively to those voxels identified as potentially non-transparent and potentially not occluded in the third and fourth steps 8, 9; and
a sixth step 11 of rendering the filtered image data for display on the display screen 4.

More detail of how these steps can be implemented in some embodiments is contained in the description below.

Described here is an approach for visualizing streaming volume data which exploits visibility information to leverage computational resources only to contributing parts of the data to enable high-performance on-the-fly data enhancement operations. An overview of the approach is depicted in FIG. 3. This illustrates how 4D data is streamed directly from the ultrasound scanner. In the first stage, a the visible set of voxels is calculated based on the opacity transfer function. Next, the neighbourhood information is evaluated and the set of potentially visible voxels is passed to the next stage. If the data does not fit into the graphical processing unit (GPU) memory, memory consumption can be optimized by performing a visibility-driven data packing before processing the data. Finally, the processed data is rendered. The pipeline receives one volume of the ultrasound stream at a time. This volume can then undergo multiple sequential processing operations and the result of these operations is then displayed by a volume rendering algorithm. The strategy for enabling on-the-fly processing of live streamed volume data is that processing operations only have to be applied to those regions which affect the final displayed image. This means that completely transparent voxels do not have to be processed, but it also means that occluded regions (regions where viewing rays have already accumulated full opacity before reaching them) can be skipped.

To facilitate this, permissible processing operations (such as filtering) should satisfy the following conditions. The input volume is a scalar-valued volumetric function ƒ: [symbol] ³→ [symbol] . In general, a processing operation g(p) replaces the original value at a voxel position p with a new value. For any such operation, the filtered function value g(p) should:
(1) only depend on values of ƒ in a finite neighbourhood Ωₚ around a position p, i.e., that it should have compact support, and
(2) satisfy g(p)ε[inf(Ωₚ), sup(Ωₚ)], i.e., the new value falls within the minimum and maximum values in that neighbourhood.

Both requirements are trivially true for any convolution with normalized weights (mean, Gaussian, etc.), but also hold for a wide range of other smoothing or de-noising operations including nonlinear filters such as the median or bilateral filters. An edge-detector, containing negative values in the operator mask, does not conform with this requirement. Without the above requirements a processing operation could, in principle, be a random number generator and there would be no way to predict the resulting visibility. In order to exactly determine the final visibility at every position, full processing and volume rendering steps would have to be performed. Given these two assumptions, however, methods embodying the invention can be used to conservatively predict the visibility of a voxel position p, prior to the application of g. This involves using a visibility prediction function v(p)ε{0,1} which, for every position p, is 0 when the application of the processing operation at that position can be safely (i.e., without changing the final image) skipped and has a value of 1 otherwise.

Ideally, this prediction function is much cheaper to compute than g itself and we can therefore not only amortize its costs but also reduce the overall processing time by only applying g to those positions that are potentially visible (i.e., where v(p)=1). We aim at finding the smallest possible potentially visible set of voxels, but at the same time we need to ensure that the additional processing does not outweigh our gains. Hence, there is a trade-off between the computational cost of evaluating v and the number of voxels that are incorrectly classified as visible. Our approach for computing v is comprised of two basic steps described below: First, we obtain inf(Ωₚ) and sup(Ωₚ) for the neighbourhood of the processing operation. We then perform a visibility pass which generates a binary volume which stores the values of v.

Based on the set of potentially visible voxels, we then generate a working set of the volume data which needs to be processed (see further below). Our pipeline uses a block-based volume layout which enables optimized memory access and volume traversal and also reduces the intermediate storage requirements for multiple processing steps. Only blocks which contain at least one non-transparent voxel need to be accessed during the processing operation. We use a virtual block table which is generated on-the-fly. After the volume has been processed, it can be rendered using a standard GPU-based volume ray-caster as briefly described further below.

We first calculate a view-dependent set of potentially visible voxels (PVV), after application of the filtering operation, which takes into account both transparency and occlusion. As our approach is designed to handle live streaming volume data, this set needs to be recomputed for every volume update. The approach described below copes with both of the following cases:

Case 1. A region in the volume considered to be an occluding region before the filtering operation has been applied may get new values which are mapped to opacity α=0 during the visualization stage. This means that it no longer acts as an occluding region. This case is illustrated in FIG. 4.

Case 2. The filtering operation can also change data values in such a manner that previously invisible regions will be non-transparent after its application. For example, a simple averaging kernel may increase the value of some voxels, while decreasing others, such that a voxel which was previously below a transparency threshold of the opacity transfer function may be above it after the averaging.

To take into account such potential visibility changes without actually executing the filtering operation on the entire image data set, we make use of the two assumptions stated in the previous section, i.e., the filter has compact support and the result of the operation lies within the minimum and maximum of its neighbourhood. To quickly determine the relevant opacity contributions for a particular neighbourhood, we generate the following two lookup tables:

$$\begin{matrix}{α}_{min} ( i , j ) = {min}_{u ∈ [ i , j ]} ( {f}_{α} ( u ) ) & ( 1 ) \\ {α}_{max} ( i , j ) = {max}_{u ∈ [ i , j ]} ( {f}_{α} ( u ) ) & ( 2 )\end{matrix}$$

where αₘ, and αₘₐₓ are the minimum and maximum, respectively, opacity values in the transfer function ƒ_{α} for all values in the interval [i, j]. Both αₘ, and αₘₐₓ can be computed simultaneously and stored in a single 2D texture as two channels. Computation of these tables is straight-forward and only consumes a negligible amount of time when the transfer function is modified. An example of an opacity transfer function and its corresponding αₘ, and αₘₐₓ are given in FIG. 5.

Determining the potentially visible voxels (PVV) then proceeds as follows:

Minimum/Maximum Computation.

In order to obtain information about the voxel neighbourhood, this needs to be recomputed for every new volume. We compute, for each position p in the volume, the minimum inf(Ωₚ) and maximum value sup(Ωₚ) with a given neighbourhood Ωₚ determined by the support of the processing operation (i.e., the size of the kernel for convolution operations). In one particular implementation, we use an OpenCL kernel in a multi-pass approach which exploits the fact that the min and max filters are separable. While this is not a cheap operation, it is still significantly less costly than the types of filtering operations which we want to support.

Visibility Evaluation.

The set of potentially visible voxels is then computed in a front-to-back visibility pass which outputs a binary volume. As one value needs to be generated for each voxel, we use axis-aligned traversal implemented as an OpenCL kernel. Similar to 2D texture-mapped volume rendering, the slice axis is chosen as the major axis of the volume most parallel to the current viewing direction. The αₘₐₓ table is used to determine the visibility of a voxel, while αₘᵢₙ is used to evaluate its contribution to the accumulated opacity in the following manner:

Aₚ_{₀}=0

Aₚ_{ᵢ}=Aₚ_{ᵢ₋₁}+(1−Aₚ_{ᵢ₋₁})·αₘᵢₙ(inf(Ωₚ_{ᵢ}),sup(Ωₚ_{ᵢ})) (3)

where Aₚ_{ᵢ} is the accumulated opacity at the i-th sample position pᵢ along a viewing ray, and inf(Ωₚ_{ᵢ}),sup(Ωₚ_{ᵢ}) are the minimum and maximum values, respectively, in the filter neighbourhood. Accumulating with αₘₐₓ ensures that all no viewing ray will be terminated too early.

The final visibility prediction function, which is the characteristic function of the PVV set, is then defined as:

$$v ( {p}_{i} ) = \{ \begin{matrix}1 & if {α}_{max} ( inf ( {Ω}_{{p}_{i}} ) , sup ( {Ω}_{{p}_{i}} ) ) > 0 and {A}_{{p}_{i - 1}} < 1 \\ 0 & otherwise\end{matrix} \qquad ( 4 )$$

While the PVV gives us information about which voxel positions are potentially visible in the final image after the processing operation has been applied, we also need to make sure that the results of the processing operation are correct. Many data enhancement techniques require information about the neighbourhood. Moreover, many approaches are carried out in an iterative fashion, e.g., when filters are separable. If we rely only on the PVV that considers reduced occlusion and increased visibility discussed so far, iterations will obtain incorrect information from the neighbourhood of the PVV in later passes.

This problem is illustrated in FIG. 6, which shows how, if data enhancement consists of one operation only, expansion of the PVV is not necessary. But if it runs in two iterations, values within the band of influence (defined below) must be processed in the first iteration, since the second iteration relies on its results If the enhancement runs in three iterations, the radius of the band of influence is larger accordingly.

To handle this situation, we dilate the visibility mask to obtain a working set of voxels (WSV). Then we define a band of influence (BOI) as WSV\ PVV. For example, similarly to the min-max computation, if a filtering operation has p iterations and in each iteration, the operation requires a neighbourhood of radius q, then the visibility mask must be dilated by a radius p×q. Using WSV for filtering calculations will ensure that the values of all voxels in PVV will be correct after the final iteration of the filter.

Many filters require additional buffers to store information. Lowest-variance filtering requires the direction along the lowest variance to be stored as a 3D vector field. Anisotropic diffusion requires an additional swap-buffer.

With larger volumes, it can be challenging to keep such buffers in GPU memory simultaneously. As we are only filtering the visible data, we only need to keep a fraction of the data on the GPU for processing. We can reduce memory usage by only storing buffers for the actually visible parts of the volume. For instance, lowest-variance filtering needs to store an additional 3D vector with float components for each voxel it processes. For the entire volume, this extends the memory requirements by a factor of 12 if the original volume is an 8-bit byte array and a 32-bit floating point representation is used for the vectors.

In the present approach, we subdivide the volume into fixed-size blocks. We then need to only store and process those blocks that contain voxels which are part of the WSV. We can then process the array one block at a time, rather than the entire volume. The packing mechanism is illustrated in FIG. 7. From the WSV we create the following data structures which are used during the execution of the filtering operations:

Block Data Pool.

The block pool is a buffer which stores the data values for all blocks in the working set. Additionally, for reasons explained below, we store one empty block (filled with zero values) at index zero. The block size is set at compile-time and for all experiments in the paper we used a block size of B_{x,y,z}=32.

Virtual Block Table.

The virtual block table stores the locations of virtual blocks indices in the block data pool. For an input volume of size Vₓ. V_{y}. V_{z}, it has a size of Tₓ. T_{y}. T_{z} with T_{x,y,z}=V_{x,y,z}/B_{x,y,z}+2. The inflated parts of the virtual block table are initialized with zero, i.e., they point to the empty block. In this way, the subsequent processing passes can safely access locations beyond the volume boundaries while avoiding costly out-of-bounds checks. Note that this inflation is different from the common duplication of voxel values within each brick, which does not occur in our approach. For blocks which are not in the current working set, the virtual block table also contains the value zero.

Working Set Array.

This array contains only the indices of blocks in the current working set. Hence, the working set array is a linearized version of the virtual block table where zero entries have been removed. It is used for volume traversal during the execution of the processing operation.

After generating the working set data structures, we can now apply the actual filtering operation. Each processing pass takes as input the block data pool and the virtual block table, and writes its result into a new buffer which duplicates the structure of the working block pool. By traversing the working set array, each block can be processed independently. The virtual block table is used to resolve the locations of neighbourhood voxels which lie outside the current block. For a voxel position x,y,z, we first compute its index i in the virtual block table

i=(x/Bₓ+1)+(y/B_{y}+1)·Tₓ+(z/B_{z}+1)·Tₓ·T_{y}

which is then used to retrieve the corresponding block's offset o in the block data pool. The final location p of the voxel in the block table pool is then

p=o+(x mod Bₓ)+(y mod B_{y})·Bₓ+(z mod B_{z})·Bₓ·B_{y}.

While our block-based representation could be directly used for rendering, the fact that we do not duplicate boundary voxels can prevent the use of native trilinear interpolation which impacts the performance. Instead, however, we can exploit the GL_ARB_sparse_texture extension in OpenGL which natively supports rendering from sparse texture representations without duplicated block borders. While this involves an additional copy operation of the visible blocks into the sparse texture, we have found that this option is preferable both in terms of performance, flexibility and ease of implementation. This approach is not tied to any specific volume rendering algorithm, and may flexibly integrate different renderers including standard GPU-based ray casting and slice-based multidirectional occlusion volume rendering. Furthermore, as block-based visibility information—as described above—is already available, it can be straight-forwardly employed for rasterization-based empty-space skipping.

The described processing pipeline has been implemented in C++ using OpenCL. The volume rendering itself was performed in OpenGL on a PC with a 3.06 GHz CPU with NVIDIA™ GTX680 GPU with 2 GB of dedicated graphics memory running Windows™ 7.

It can be shown that this approach for culling invisible regions of the volume is correct in the sense that it does affect the final image.

FIGS. 8 and 9 profile the performance of our method on exemplary data. They illustrate a performance boost of visibility-driven processing compared to full processing with the lowest-variance filtering with radius 5 on a stream of 3D cardiac ultrasound datasets (128×100×128). FIG. 9 illustrates the behaviour of the same filter for the different dataset sizes: the same cardiac stream as in FIG. 8: a streamed 3D ultrasound of a fetus (229×261×114) and streamed 3D ultrasound of a gall bladder (200×201×141).

FIG. 8 shows the performance of the lowest-variance filter. The black horizontal line shows the constant time that is needed to process the full volume. The blue line shows the dependency of the performance of the visibility-driven filtering on the amount of visible data, using a trivial set of visible voxels (trivial VV). This means that this set of voxels was defined only based on the immediate occlusion and opacity of the voxels, but not on the eventual change during filtering as we described above. The red line shows the performance with respect to the amount of visible data, but using the correct PVV as described above. We observe that the difference between the blue and the orange line is approximately constant and relatively small, also for other datasets for which we do not display the performance curves. Therefore, we can conclude that the correct PVV computation does not represent a significant performance overhead in the entire pipeline. Only if more than 85% of the data is visible, which is most likely not the case for cardiac 3D ultrasound, the visibility-driven filtering optimization would no longer pay off. We also measured performance using other filters, e.g., Perona-Malik anisotropic diffusion and bilateral filtering and they showed a very similar trend as lowest-variance filtering. FIG. 9 plots the performance gain for different ultrasound datasets with sizes using lowest-variance filtering.

This visibility-driven filtering requires a certain amount of supporting buffers which consume memory. This could become an issue when handling larger datasets than ultrasound datasets used in obstetrics or 3D echocardiology. For datasets that do not fit into the memory together with their supporting buffers, visibility-driven data packing will become necessary. In FIG. 8, the green line plots the performance dependency if we use the data packing scheme with blocks. Obviously, the data packing scheme does not pay off for small datasets, but it is inevitable for large datasets that do not fit into the GPU memory together with the supporting buffers.

On modern ultrasound scanners with a 4D cardiac probe, ultrasound volumes are acquired at rates of 10-15 volumes per second, depending on the spatial extent of the acquired volume (sector). The larger the sector, the bigger is the volume and the lower is the acquisition rate. According to our own experience, larger sectors are acquired at ca. 15 fps. In one example, filtering all voxels took 0.076 seconds, whereas filtering only the PVV set took 0.045 seconds, which allows higher frame rates to be supported.

The approach described above results in a considerable reduction of processing times for realistic percentages of visible voxels and hence enables real-time filtering and, consequently, high-quality visualization of streaming volume data in cases where this was previously impossible.

1. A method of processing three-dimensional image data comprising a plurality of voxels to generate processed image data suitable for volume rendering, from a viewpoint, using an opacity transfer function, wherein the method comprises:
applying a lower-bound-generating function to the image data to generate a lower bound for each voxel in the image data;
applying an upper-bound-generating function to the image data to generate an upper bound for each voxel in the image data;
for each voxel in the image data, determining whether there exists:
(i) a value, between the lower bound and the upper bound for that voxel, at which the voxel is not completely transparent under the opacity transfer function; and
(ii) a set of values for a set of sample points lying along or adjacent a viewing ray between the viewpoint and said voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray; and
applying a predetermined processing operation to those voxels for which both determinations are true, to generate processed image data,
wherein the bound-generating functions and the predetermined processing operation are such that, for any three-dimensional image data, the value of a voxel after the processing operation is applied to the image data will always be between the lower bound and the upper bound given by the bound-generating functions when applied to that voxel.

2. The method of claim 1, wherein the lower-bound-generating function and the upper-bound-generating function are such that a number of processor clock cycles required to evaluate both functions for a voxel is less than half a number of processor clock cycles required to apply the processing operation to the voxel.

3. The method of claim 1, wherein the processing operation is not applied to a majority of the voxels for which one or both of the determinations are false.

4. The method of claim 1, wherein the processing operation is applied only to those voxels for which both determinations are true and to a band of voxels surrounding these voxels.

5. The method of claim 1, wherein the processing operation has an output at any given position that depends only on values in a finite neighbourhood around that position.

6. The method of claim 5, wherein the lower-bound-generating function and the upper-bound-generating function have outputs at any given position that depend only on values in the same said finite neighbourhood around the given position.

7. The method of claim 1, wherein the lower-bound-generating function, applied to a position, outputs the lowest value in a finite neighbourhood around that position, and the upper-bound-generating function, applied to a position, outputs the highest value in the finite neighbourhood around that position.

8. The method of claim 1, wherein the step of determining whether there exists a value, between the lower bound and the upper bound for a voxel, at which the voxel is not completely transparent under the opacity transfer function, comprises determining the maximum opacity value attained by the opacity transfer function across an interval between the lower bound and the upper bound for the voxel, and determining whether the voxel is completely transparent at this opacity value.

9. The method of claim 1, wherein the step of determining whether there exists a set of values for a set of sample points lying along or adjacent a viewing ray between the viewpoint and a voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray, comprises accumulating opacity values of sample points along a viewing ray to the voxel, where each opacity value is the minimum opacity value attained by the opacity transfer function across the interval between the lower bound and the upper bound for the sample point, and determining whether the voxel is not completely occluded with these accumulated opacity values.

10. The method of claim 1, comprising pre-computing a set of maximum opacity values for a set of possible voxel value intervals.

11. The method of claim 1, comprising pre-computing a set of minimum opacity values for a set of possible voxel value intervals.

12. The method of claim 1, wherein the processing operation comprises any one or more of the following operations: a smoothing filter, a noise-reducing filter, a mean filter, a median filter, a Gaussian smoothing filter, a contrast-enhancing filter, a Perona-Malik anisotropic diffusion, a bilateral filtering, and a segmentation operation.

13. The method of claim 1, comprising receiving a sequence of two or more frames of three-dimensional image data, applying the recited steps to each frame of image data, and displaying rendered video output from the processed image data.

14. An apparatus comprising:
an input for receiving three-dimensional image data comprising a plurality of voxels; and
processing logic, arranged:
to apply a lower-bound-generating function to the image data to generate a lower bound for each voxel in the image data;
to apply an upper-bound-generating function to the image data to generate an upper bound for each voxel in the image data;
for a predetermined opacity transfer function and a predetermined viewpoint, to determine, for each voxel in the image data, whether there exists:
(i) a value, between the lower bound and the upper bound for that voxel, at which the voxel is not completely transparent under the opacity transfer function; and
(ii) a set of values for a set of sample points lying along or adjacent a viewing ray between the viewpoint and said voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray; and
to apply a predetermined processing operation to those voxels for which both determinations are true, to generate processed image data suitable for volume rendering from the viewpoint,
wherein the bound-generating functions and the predetermined processing operation are such that, for any three-dimensional image data, the value of a voxel after the processing operation is applied to the image data will always be between the lower bound and the upper bound given by the bound-generating functions when applied to that voxel.

15. (canceled)

16. The apparatus of claim 14, further comprising a display screen for displaying a rendered view derived from the processed image data.

17. The apparatus of claim 14, wherein the processing logic is arranged to apply the processing operation only to those voxels for which both determinations are true and to a band of voxels surrounding these voxels.

18. The apparatus of claim 14, wherein the processing logic is arranged to determine whether there exists a value, between the lower bound and the upper bound for a voxel, at which the voxel is not completely transparent under the opacity transfer function, by determining the maximum opacity value attained by the opacity transfer function across an interval between the lower bound and the upper bound for the voxel, and determining whether the voxel is completely transparent at this opacity value.

19. The apparatus of claim 14, wherein the processing logic is arranged to determine whether there exists a set of values for a set of sample points lying along or adjacent a viewing ray between the viewpoint and a voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray, by accumulating opacity values of sample points along a viewing ray to the voxel, where each opacity value is the minimum opacity value attained by the opacity transfer function across the interval between the lower bound and the upper bound for the sample point, and determining whether the voxel is not completely occluded with these accumulated opacity values.

20. The apparatus of claim 14, wherein the processing operation comprises any one or more of the following operations: a smoothing filter, a noise-reducing filter, a mean filter, a median filter, a Gaussian smoothing filter, a contrast-enhancing filter, a Perona-Malik anisotropic diffusion, a bilateral filtering, and a segmentation operation.

21. A non-transitory computer-readable medium comprising software instructions which, when executed by a processing system, causes the processing system:
to apply a lower-bound-generating function to three-dimensional image data, comprising a plurality of voxels, to generate a lower bound for each voxel in the image data;
to apply an upper-bound-generating function to the image data to generate an upper bound for each voxel in the image data;
for a predetermined opacity transfer function and a predetermined viewpoint, to determine, for each voxel in the image data, whether there exists:
(i) a value, between the lower bound and the upper bound for that voxel, at which the voxel is not completely transparent under the opacity transfer function; and
(ii) a set of values for a set of sample points lying along or adjacent a viewing ray between the viewpoint and said voxel, each value of the set being between a lower bound and an upper bound for the respective sample point, according to the lower- and upper-bound-generating functions for the sample point, wherein the set of values is such that said voxel is not completely occluded from the viewpoint when the opacity transfer function is applied to the sample points along the viewing ray; and
to apply a predetermined processing operation to those voxels for which both determinations are true, to generate processed image data suitable for volume rendering from the viewpoint,
wherein the bound-generating functions and the predetermined processing operation are such that, for any three-dimensional image data, the value of a voxel after the processing operation is applied to the image data will always be between the lower bound and the upper bound given by the bound-generating functions when applied to that voxel.
